(19) United States
(12) Patent Application Publication
(10) Pub. No.: US 2026/0232276 A1
(43) Pub. Date: Aug. 13, 2026

(54) PATIENT POSITIONING FOR MEDICAL IMAGE ACQUISITION

(71) Applicant: Siemens Healthineers AG, Forchheim (DE)

(72) Inventors: Rishabh SHAH, Wentworth Point (AU); Daphne YU, Yardley, PA (US); Kaloian PETKOV, Lawrencevillle, NJ (US); Sophia Katharina HEROLD, Maktzeuln (DE); Ralf NANKE, Neunkirchen am Brand (DE); Gerben TEN CATE, Hausen OT Wimmelbach (DE); Steffen KAPPLER, Hallerndorf-Pautzfeld (DE)

(73) Assignee: Siemens Healthineers AG, Forchheim (DE)

(21) Appl. No.: 19/538,239

(22) Filed: Feb. 12, 2026

(30) Foreign Application Priority Data
Feb. 13, 2025 (DE) 10 2025 105 347.2

(51) Int. Cl. A61B 5/00; A61B 5/055

(52) U.S. Cl. CPC A61B 5/70; A61B 5/0077; A61B 5/055; A61B 5/744; A61B 5/7475

(57) ABSTRACT

A method for patient positioning for medical image acquisition, comprises: determining a machine-interpretable pose based on a selected specific human readable target pose template using a pose interpreter; determining a complete target pose of an avatar based on the determined machine-interpretable pose and based on an avatar model including anatomical constraints using an anatomical pose resolver, thereby resolving physically impossible intersections and collisions of body parts; rendering a fully posed avatar target based on the complete target pose of an avatar; presenting the fully posed avatar target using a human-machine interface; detecting the current patient pose of the patient using a non-radiation image capture unit; comparing the fully posed avatar target with the detected current patient pose of the patient; and determining an adjustment measurement based on the result of the comparison.

### CROSS-REFERENCE TO RELATED APPLICATION(S)

The present application claims priority under 35 U.S.C. § 119 to German Patent Application No. 10 2025 105 347.2, filed Feb. 13, 2025, the entire contents of which is incorporated herein by reference.

### FIELD

One or more example embodiments of the present invention relate to a method for patient positioning for medical image acquisition. Further, one or more example embodiments of the present invention concern a patient positioning system.

### BACKGROUND

Medical image acquisition requires a precise positioning of a patient in specific poses in order to properly capture internal anatomical structural details. Clinical practice reference descriptions are available as guidelines to educate and train medical image equipment technicians (by short “technicians”). Hence, correct patient positioning is learned via reference textbooks and training courses as part of technician training. Usually the technicians have the reference guidelines available for reference during the exams, but even then it is often difficult to judge the accuracy of the positioning of the patient. The large body of references are described in free text or photos that technicians must memorize and adapt to individual patient situations, which may result in poor image acquisition that may need to be repeated upon review. There may be variable adaptation of the guideline to specific patient mobility constraint situations that are difficult to learn. Therefore, it is still difficult to achieve quality and consistency due to the wide variety of poses that need to be learned and possible skilled staff shortage.

In the worst case, the image may not be usable by the subsequent radiological reading and patient may need to be rescheduled for repeating the visit. This results in additional discomfort and a larger radiation dose for the patients.

Hence, there is a problem of achieving a high quality medical imaging result together with lower radiation dose for the patients, a reduced need of skilled persons and improved comfort for the patients compared to the currently existing state of the art.

### SUMMARY

At least the aforementioned problem is solved by a method for patient positioning for medical image acquisition and by a patient positioning system as claimed.

According to the method for patient positioning for medical image acquisition, according to one or more example embodiments of the present invention, a special type of a scan required for the patient is selected. In particular, such a type of a scan may include the focus of a special body region or a special angle of view for image acquisition. Preferably, medical image acquisition comprises imaging based on computer tomography or magnetic resonance tomography.

Further, a specific human readable target pose template that defines the patient target pose for the selected scan required for the patient is generated. The generation of the specific human readable target pose template also includes the determination of a body region to be visualized.

Furthermore, a machine-interpretable pose based on the selected specific human readable target pose template using a pose interpreter is determined. Besides, a complete target pose of an avatar is determined based on the determined machine-interpretable pose and additionally based on an avatar model (e.g., a pre-stored avatar model) including anatomical constraints using an anatomical pose resolver, wherein physically impossible intersections and collisions of body parts are resolved.

Then, a fully posed avatar target is rendered based on the complete target pose of an avatar. Further, the fully posed avatar target is displayed to the user, in particular the technician, using a human-machine interface. Additionally, a detected current patient pose is detected using a non-radiation image capture unit. The non-radiation image capture unit preferably comprises an optical camera which is directed to the patient. In particular, the non-radiation image capture unit captures an image of the patient from a predetermined view angle and with a predetermined distance between the non-radiation image capture unit and the patient. Further, the current patient pose is determined based on these camera parameters and the captured image of the patient.

Then, the fully posed avatar target is compared with the detected current patient pose. Finally, an adjustment measurement is determined based on the result of the comparison between the fully posed avatar target and the detected current patient pose.

The method, according to one or more example embodiments of the present invention, for patient positioning for medical image acquisition enables a patient positioning assistant, where an avatar of the target patient pose is presented to visually guide the technician to physically move the patient into the correct position relative to the imaging equipment. The target patient poses are interpreted from literature free text guidelines, and converted to a structured set of human-and machine-readable poses that drive the avatar positioning. The result is a quantitative representation of each position, scale, rotational angle, etc. for all represented parts and joints of the target model avatar body skeleton, i.e. the fully posed avatar target, that can be used for comparisons and to produce the scanner parameters for image acquisition. The scanner is augmented with a camera-based patient pose detector such that more specific guiding instructions can be given to the technician or patient in moving from incorrect to reference position.

The patient positioning system comprises a selection unit for selecting a special type of a scan required for a patient. Further, the patient positioning system includes an anatomically aware large language model unit for generating a specific human readable target pose template that defines the patient target pose for the selected special type of a scan required for the patient. Large language models, by short LLMs, can respond to free-text queries without being specifically trained in the task in question. LLMs are often used in healthcare settings.

An LLM is a type of a computational model designed for natural language processing tasks such as language generation. As language models, LLMs acquire these abilities by learning statistical relationships from vast amounts of text during a self-supervised and semi-supervised training process.

The largest and most capable LLMs are artificial neural networks built with a decoder-only transformer-based architecture, enabling efficient processing and generation of large-scale text data. Modern models can be fine-tuned for specific tasks or guided by prompt engineering. These models acquire predictive power regarding syntax, semantics, and ontologies inherent in human language corpora.

The patient positioning system also includes a pose interpreter for determining a machine-interpretable pose based on the selected specific human readable target pose template. The pose interpreter takes language-based definitions of the relationships between body parts and converts them to positions, rotations, parameters, etc. for a 3D skeletal rig, which is used to animate the patient avatar, i.e. the fully posed avatar target.

Furthermore, the patient positioning system comprises an anatomical pose resolver for determining a complete target pose of an avatar based on the determined machine-interpretable pose and additionally based on an avatar model (e.g., a pre-stored avatar model) including anatomical constraints using an anatomical pose resolver, wherein physically impossible intersections and collisions of body parts are resolved.

Also a rendering unit for rendering a fully posed avatar target based on the complete target pose of an avatar is part of the patient positioning system, according to one or more example embodiments of the present invention.

Besides, the patient positioning system comprises a human-machine interface for presenting the fully posed avatar target and a pose detection unit for detecting the actual pose of the patient, i.e. the current patient pose, using a non-radiation image capture unit.

The patient positioning system further comprises a comparison unit for comparing the generated fully posed avatar target with the detected actual pose of the patient, i.e. the current patient pose, and an adjustment unit for determining an adjustment measurement based on the result of the comparison. The adjustment measurement preferably comprises an adaption of the patient position and/or an adaption of the acquisition parameters of the planned medical image acquisition. The patient positioning system shares the advantages of the method for patient positioning for medical image acquisition, according to one or more example embodiments of the present invention.

Some units or modules of the system mentioned above can be completely or partially realized as software modules running on a processor of a respective computing system, e.g. of a control device of a medical imaging system. A realization largely in the form of software modules can have the advantage that applications already installed on an existing computing system can be updated, with relatively little effort, to install and run these units of the present application. At least one object of one or more example embodiments of the present invention is also achieved by a computer program product with a computer program that is directly loadable into the memory of a computing system, and which comprises program units to perform the steps of the inventive method, in particular the step of selecting a type of scan required for the patient, the step of selecting a specific human readable target pose template that defines the patient target pose for the selected type of scan, the step of determining a machine-interpretable pose based on the selected specific human readable target pose template using a pose interpreter, the step of determining a complete target pose of an avatar, the step of rendering a fully posed avatar target based on the complete target pose (CTP) of an avatar, the step of presenting the fully posed avatar target to a user using a human-machine interface, the step of detecting the current patient pose of the patient using a non-radiation image capture unit, the step of comparing the fully posed avatar target with the detected current patient pose of the patient and the step of determining an adjustment measurement based on the result of the comparison, when the program is executed by the computing system. In addition to the computer program, such a computer program product can also comprise further parts such as documentation and/or additional components, also hardware components such as a hardware key (dongle etc.) to facilitate access to the software.

A non-transitory computer readable medium such as a memory stick, a hard-disk or other transportable or permanently-installed carrier can serve to transport and/or to store the executable parts of the computer program product so that these can be read from a processor unit of a computing system. A processor unit can comprise one or more microprocessors or their equivalents.

The dependent claims and the following description each contain particularly advantageous embodiments and developments of the present invention. In particular, the claims of one claim category can also be further developed analogously to the dependent claims of another claim category. In addition, within the scope of the present invention, the various features of different exemplary embodiments and claims can also be combined to form new exemplary embodiments.

In a variant of the method for patient positioning for medical image acquisition, according to one or more example embodiments of the present invention, the adjustment measurement comprises a proposal for physical adjustment of the pose of the patient and displaying the proposal using the human-machine interface. Advantageously, not only the fully posed avatar target, but also a corrected current patient pose is displayed such that a technician is able to determine if a proposed correction of the current patient pose is appropriate

In a preferred variant of the method for patient positioning for medical image acquisition, according to one or more example embodiments of the present invention, the adjustment measurement includes
determining adjusted scan projection parameters of a medical imaging system used for the medical image acquisition and
adjusting the medical imaging system based on the adjusted scan projection parameters.

Advantageously, the medical imaging system is adapted to an actual position of a patient, for example in case the patient is not able to accept the target position due to individual anatomical constraints or due to a special injury or illness.

Preferably, the step of determining adjusted scan projection parameters includes the sub-step of calculating an adjusted scan projection based on the fully posed avatar target and/or based on the detected current patient pose of the patient. Hence, the scan projection is adapted to the fully posed avatar target or to the detected current patient pose of the patient.

In a preferred arrangement of the method, according to one or more example embodiments of the present invention,
an amendment information is received by the human-machine interface for amending the pose of the fully posed avatar target such that a corrected fully posed avatar target is generated and
the steps of comparing the fully posed avatar target with the detected current patient pose of the patient and determining an adjustment measurement based on the result of the comparison are carried out based on the corrected fully posed avatar target.

Advantageously, the fully posed avatar target can be adjusted by a technician being involved in a medical imaging of a patient.

Also preferably, the corrected fully posed avatar target is analyzed using a target interpreter and based on avatar models (e.g., pre-stored avatar models), wherein a corrected specific human readable target pose template is generated. Advantageously, a human readable target pose template can be adapted to a corrected fully posed avatar target, for example for an individual patient or a special type of imaging task.

Further preferred, the specific human readable target pose template is automatically generated using an anatomical aware large language model applied to a clinical position guideline description. Advantageously, the specific human readable target pose template can be automatically determined based on a text based guideline description.

In a preferred embodiment of the method, according to the present invention, the avatar model is specified based on at least one of the following features concerning anatomical characteristics:
gender,
age,
body size,
shape,
height,
weight,
specific anatomical constraints,
maximum joint rotation angulation,
reach of extremities.

Advantageously, the avatar is adapted to available knowledge about a patient.

In a preferred variant of the method, according to one or more example embodiments of the present invention, specific constraints for a body part used for generating an avatar model are amended based on the knowledge of individual disease anomalies of a patient.

Also preferred, the avatar model comprises a set of avatar models including base poses, typically used for medical image acquisitions preferably comprising at least one of the following types of default poses:
supine acquisition,
oblique laying pose on a scanning table,
standing pose,
sitting pose.

Advantageously, the basic schemes of poses of avatars are already saved and available for generating a fully posed avatar target.

In a preferred embodiment of the method, according to the present invention, the step of determining a machine-interpretable pose based on the selected specific human readable target pose template comprises the substep of calculation of one of the following parameters:
specific scale of each of the avatar body part and skeleton joints,
angulation of each of the avatar body part and skeleton joints,
position of each of the avatar body part and skeleton joints, to give the machine precise positioning.

Advantageously, based on a human readable text, physical parameter values for calculating an individual pose of an avatar are determined.

Preferably, the step of determining a complete target pose of an avatar is performed using one of the following techniques:
iterative inverse kinematics and collision detection techniques,
learned ai models from actual human pose data.

Advantageously, all the restraints and individual patient data can be used for calculating the avatar.

Also preferred, the human-machine interface comprises the display of a comparison of the current patient pose with the target pose to adjust the position of the patient, preferably including one of the following information:
distance between the current and target position of the joints, in particular distance between the feet of the different poses,
difference of angle between current and target orientation of the joints, in particular angle difference between the knee rotation of the difference poses.

Advantageously, information for correcting a pose of a patient is visualized to a technician such that the technician is able to correct the pose of the patient until the current patient pose of the patient is identical to the visualized target pose.

In a variant of the method, according to one or more example embodiments of the present invention, the human-machine interface comprises the display of a simulated X-ray visualization of the current patient pose using image-based differentiable rendering, wherein the simulated image is compared to the desired scan image.

### BRIEF DESCRIPTION OF THE DRAWINGS

The present invention is explained below with reference to the figures enclosed once again. The same components are provided with identical reference numbers in the various figures.

The figures are usually not to scale.

FIG. 1 shows a schematic view on a single avatar from two different angles of view, in the target pose for hip evaluation with pelvic X-ray,

FIG. 2 shows a schematic diagram illustrating a method and a system, according to one or more example embodiments of the present invention,

FIG. 3 shows a schematic view on a visual guidance in foot rotation from current position to target position, according to an embodiment of the present invention,

FIG. 4 shows a schematic view on a correction of the poses of the legs of a patient, according to an embodiment of the present invention,

FIG. 5 shows a flow diagram illustrating a method for patient positioning for medical image acquisition according to an embodiment of the present invention,

FIG. 6 shows a schematic view on a patient positioning system according to an embodiment of the present invention.

### DETAILED DESCRIPTION

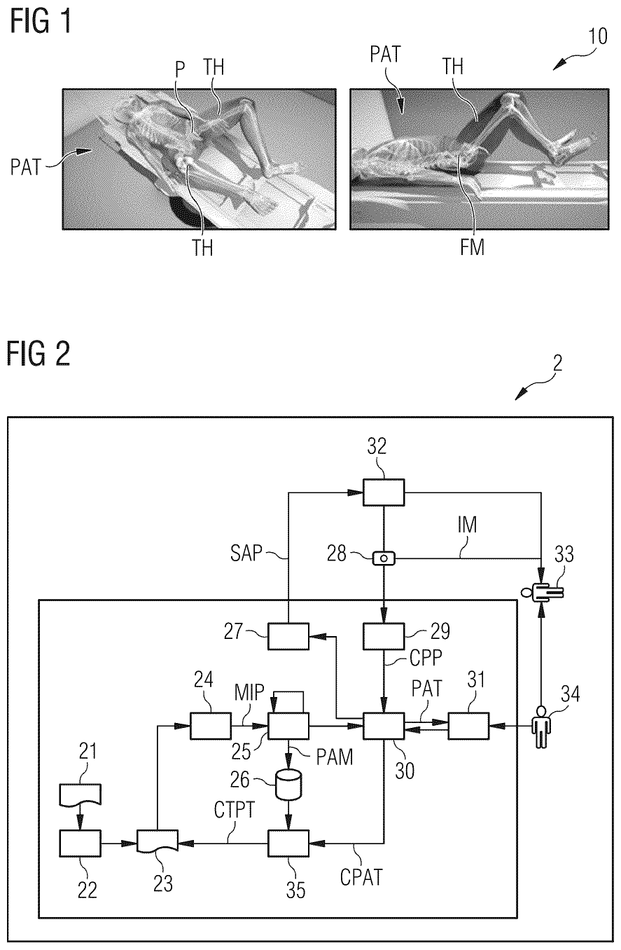

In FIG. 1, a schematic view 10 on a human-machine interface of a patient position assistance using an avatar for providing technician visual of a target patient position for medical image acquisition derived using the described technique from a reference position text description is shown.

In this example, the guideline text used is “Supine centered to CR und IR (“CR” is an abbreviation of “central ray” and “IR” is an abbreviation for “image receptor”), flex hips and knees and abduct both thighs TH equally to 45 degree from vertical if possible, with feet together. Ensure no rotation of pelvis. Center IR to CR. CR perpendicular to level of femoral heads. SID 40-44 inches”. Hence, in FIG. 1, the left view is a perspective view on a fully posed avatar target PAT and the right view is a side view on that fully posed avatar target PAT. The fully posed avatar target shows both thigs TH equally abducted to 45 degree from vertical with a not rotated pelvis P. The patient is posed in supine and centered to central ray and image receptor. Central ray CR is perpendicular to level of femoral heads FM. “SID” is an acronym for “source to image distance” and is one of the detector setting the technician needs to account for.

In FIG. 2, a schematic diagram 2 is depicted, visualizing the method for patient positioning for medical image acquisition, according to an embodiment of the present invention using a patient positioning system according to an embodiment of the present invention.

The method starts by loading a human readable target pose template 23 that defines the patient target pose for a given body region of focus, for example the legs, feet and hip shown in FIG. 1, based on the purpose of the imaging exam. For example, the reference free-text description guideline 21 for effective diagnosis of knee bone fractures may clearly describe the recommended way, a patient should position and rotate their legs, feet, hips and torso to achieve these desired angles based on anatomical knowledge of the human body. The structured target pose template scheme is defined as part of the patient positioning system definition based on analyzing a large body of reference free-text description guidelines 21. The free-text description is converted to a structured format described here as structured human-readable target pose templates 23. The format for these templates is pre-defined and consistent while maintaining the conciseness of descriptions from the free-text guidelines. These structured human-readable target pose templates 23 produce a deterministic pose result for a given avatar.

To accelerate the creation of such structured human-readable target pose templates 23, an anatomically aware large language model 22 can be used to translate from reference free text description guidelines 21 to the target pose template scheme 23. A conventional state-of-the-art large language model can be used to varying degrees of precision, or a custom trained model can be trained from existing bodies of clinical knowledge to improve the clinical terminology tokenization accuracy. As this is just a development acceleration method in lieu of manually translating the guideline to the structured target pose template 23, this can be optionally done offline (see the boxes 21, 22 in the diagram). In an embodiment, one or all of the structured target pose templates 23 can be constructed manually by an expert instead of an automated system. As the format for these target pose templates 23 is fixed, guidelines 21 from various free-text sources can be compiled into consistent target pose templates 23 compatible with a pose interpreter 24, thereby increasing the scalability of the method.

Avatar models from an avatar database 26 based on parameters affecting anatomical differences, such as gender, age, body size, shape, height, weight, are supplied with the system. In an embodiment, a set of morphs are provided for the technician 34 to configure bespoke avatars specific to a patient 33 at runtime. In another embodiment, the avatars are configured automatically based on patient information as described above. For each patient type avatar, different anatomical constraints are also built into the base avatar model (e.g., base pre-stored avatar model) PAM based on anatomical knowledge. Constraints such as maximum joint rotation angulations, reach of extremities may depend on each model's anatomical parameters such as above. Specific constraints for a body part may also be overridden by a given template to implement specializations for certain disease anomalies such as arthritis or injuries. Each avatar model PAM within the avatar database 26 is preconfigured with base poses that fully express the default poses typically used for medical image acquisitions, such as a supine or oblique laying pose on a scanning table or a standing or sitting pose.

At runtime, each structured target pose template 23 is to be used to pose the guiding avatar visualization, i.e. the posed avatar target PAT on a human-machine interface 31. The target pose template 23 is digitally loaded into the position guidance assistant system, i.e. the patient positioning system, and the semantics are interpreted by the pose interpreter 24 whose task is to calculate the relative pose description expressed in anatomical terms into a machine-interpretable pose MIP. This step includes calculation of specific scale, angulation, position of each of the avatar body part and skeleton joints to give the machine precise positioning. Note that the human readable target pose template 23 may not fully express all body parts values as required by the machine interpretation. For example, if the goal of a template is to position the left foot, pose for head, arms may not be expressed at all in the template.

After the target pose template 23 is interpreted to a set of machine-interpretable poses MIP for a given avatar (based on patient type), the avatar PAM from the avatar database 26 and the partial set of parameters and constraints are to be processed by an anatomical pose resolver 25, whose task is to resolve the full set of template specifications and constraints such that the pose follows an actual plausible pose of a human body, resolving any physically impossible intersections and collisions of body parts. This can be achieved using iterative inverse kinematics and collision detection techniques, alternatively, learned AI models from actual human pose data can also be used.

The fully posed avatar target PAT is then rendered by a computation unit 30 and presented by the human-machine interface 31 of the patient positioning system. The human-machine interface 31 is aimed to instruct a medical scanner technician 34 or patient 33 on the proper pose for the target exam. Besides displaying the posed avatar PAT, the human-machine interface 31 can also incorporate and compare the current patient pose CPP with the target pose PAT and present information useful for the technician 34 or patient 33 to adjust position, based on parameters and measurements deemed important in the reference free-text description guidelines 21, such as distance between the feet, or angle of knee rotation.

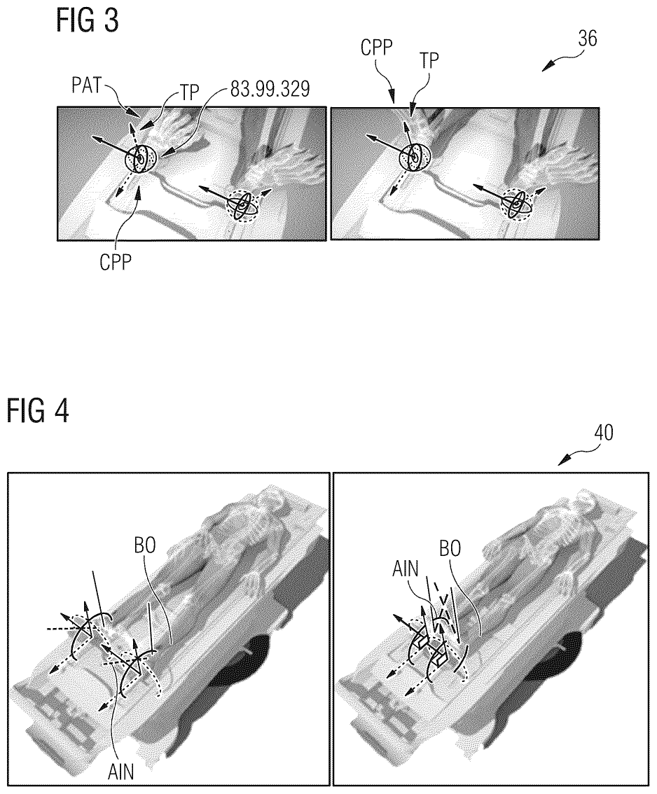

Detection of the current patient pose CPP can be achieved using a non-radiation conventional image capture unit, i.e. a camera 28, observing the patient 33 in the physical world, preferably a depth sensing camera should be used as the camera 28 for better accuracy. A pose detection unit 29 taking the camera output images IM can be used to detect an estimated current body skeleton pose or current patient pose CPP such that a digital comparison can be made. FIG. 3 shows one visual technique where target T and current pose CPP depicted on the current patient avatar PAT can be displayed on the human-machine interface 31 as information assisting the technician 34 or patient 33 to make adjustments ADJ. Many other forms of visual feedback are possible and can be rendered based on workflow preferences.

In an illustration 36 in FIG. 3, a visual guidance in foot rotation from current position to target position is shown. The left image shows a foot of the avatar depicting the current patient foot position as detected from the camera. The dotted line (former green line) in this case is the visual presentation chosen to show the current foot rotation of 83.99 degree of the right foot. The left foot is correctly posed. In the right image, when the right patient foot is actually rotated to meet the target foot pose within tolerance, the display shows a zero-degree difference to the target and therefore reached the target.

Since patient current pose CPP and target pose TP information are available using this method in quantitative form, it is possible also to use this comparison to compute the scanner acquisition parameters SAP (depicted in FIG. 2), as well as to trigger the scanner image acquisition. The scan projection calculator 27 (depicted in FIG. 2) typically requires incorporation of the target field of view information, distance, internal collimation calibration and detector geometry to calculate the projection.

In an embodiment, the visual guidance can be enhanced with simulated X-ray visualization based on the current patient pose CPP using image-based differentiable rendering. This simulated image is compared to the desired scan image if available from the guideline source using metrics such as Structural Similarity (SSIM). Gradient descent or other optimization methods (including machine learning methods) then refine the patient pose to minimize the measured image error. The X-ray image generation may use methods, including AI-based approaches, that simulate complex X-ray physics effects such as attenuation and scattering. In an example implementation, the skeleton model is derived from a reference or patient-specific CT scan and is rigged so that it can be animated together with the outer surface of the patient avatar target PAT. In further embodiments, our system displays the skeleton as an overlay or merged with the avatar, i.e. the posed avatar target PAT, rendering for visual guidance.

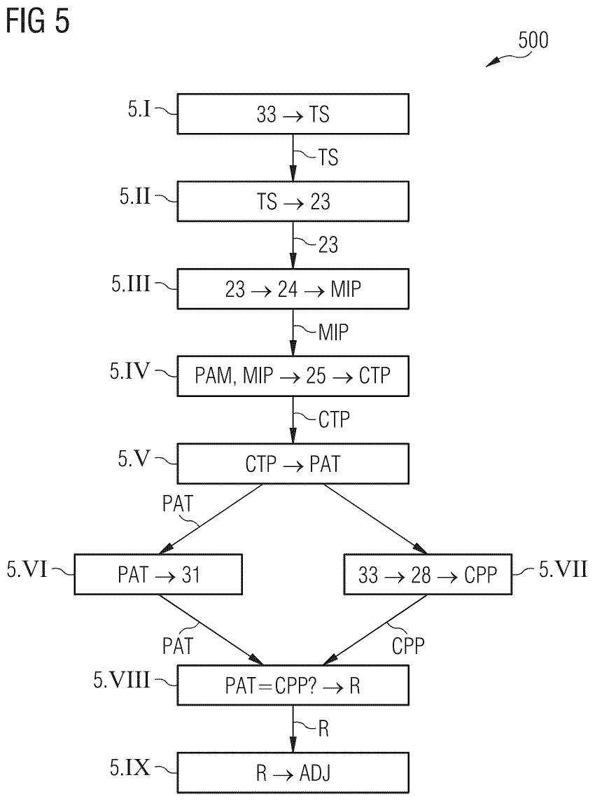

As shown in the illustration 40 of FIG. 4, in an embodiment, when camera tracking is available, color-coded visualization (in FIG. 4, colors are simulated with dotted line and dashed line) is used to give a quick indicator of patient-to-template alignment quality i.e. green for good alignment, red for unaligned. The indicator be displayed onto the outer surface of the patient avatar PAT, or on internal structures, including the skeleton and organs (e.g., modeled from reference or patient-specific CT and MR scans). The visualization of FIG. 4 illustrates a patient alignment quality. The ankle positions and orientations in the left image are far away from the template targets. Angle indicators AIN are highlighted in red (simulated as dotted line). The right patient model is aligned to the template. Angle indicators AIN are highlighted in green (simulated as dashed line). This should be for a scan requiring the feet to be turned inward, as in the image on the right, which indicates the correct pose using the green line indicators (simulated as dashed line). Bone alignment BO is also visualized in FIG. 4, in particular for the legs.

In some scanner workflows, it is also desirable to allow the end user to save derived target pose templates 23 for institutional preferences, or to create derived constraints to address certain category of patient type limitations, e.g. a modification to template specified joint rotation when a fracture is expected. In this case, it is likely that the derived constraint is defined using the human-machine interface 31 presenting from the internal machine interpreted pose representation on a current patient. In order for this derived target pose template 23 to be useable for other patient types (and avatar models), it is necessary to interpret the new pose and constraints back to the human readable target pose template 23. This is performed by the target interpreter module 35 where each defined body parts available from the base model in the template schema is compared with the derived pose and constraints. For the difference in values, the relative difference in angulation, position, scale etc. are computed and stored back to the template 23 as relative specifications such that it can be persisted and re-applied to other avatar model types.

In an embodiment, a review step may be added after anatomical pose resolver 25 and incorrect poses PAT may be manually corrected once by an expert using an avatar posing interface in software or using camera-based patient tracking; the corrected fully posed avatar target CPAT is then processed back to a human-readable target pose template 23, i.e. a corrected specific human-readable target pose template CTPT using target interpreter 35. This corrected pose template CTPT can be used in the future exams without need for corrections. In another embodiment, the system can store a patient specific avatar pose at the time of an exam to allow matching it for subsequent exams when required.

In FIG. 5, a flow diagram 500 illustrating a method for patient positioning for medical image acquisition according to an embodiment of the present invention, is shown.

In step 5.I, a type of a scan TS required for the patient 33 is selected.

In step 5.II, a specific human readable target pose template 23 that defines the patient target pose for the required scan TS is selected.

In step 5.III, a machine-interpretable pose MIP based on the selected specific human readable target pose template 23 using a pose interpreter 24, is determined.

In step 5.IV, a complete target pose CTP of an avatar is determined based on the determined machine-interpretable pose MIP and additionally based on an avatar model (e.g., a pre-stored avatar model) PAM including anatomical constraints using an anatomical pose resolver 25, wherein physically impossible intersections and collisions of body parts are resolved.

In step. 5.V, a fully posed avatar target PAT is rendered based on the complete target pose CTP of an avatar.

In step 5.VI, the fully posed avatar target PAT is presented using a human-machine interface 31.

In step 5.VII, an actual pose i.e. a current patient pose CPP of the patient 33 is detected using a non-radiation image capture unit 28.

In step 5.VIII, the fully posed avatar target PAT is compared with the detected actual pose i.e. the current patient pose CPP of the patient 33 and a result R of comparison is determined.

In step 5.IX, an adjustment measurement ADJ is determined based on the result R of the comparison.

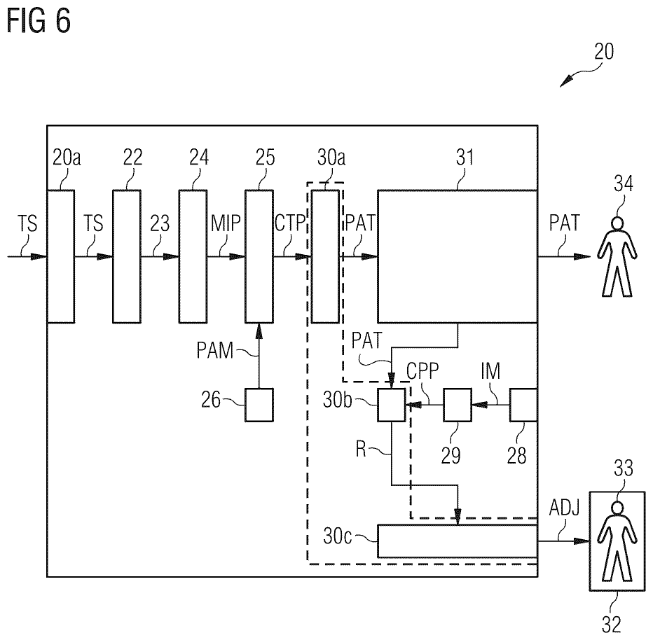

In FIG. 6, a block diagram illustrating a patient positioning system 20 according to an embodiment of the present invention, is shown.

The patient position system 20 comprises a selection unit 20a for selecting a type of scan TS required for a patient 33.

Part of the patient positioning system 20 is also an anatomically aware large language model unit 22 for generating a specific human readable target pose template 23 that defines the patient target pose for the selected type of scan TS.

Further, the patient positioning system 20 comprises a pose interpreter 24 for determining a machine-interpretable pose MIP based on the selected specific human readable target pose template 23.

The patient positioning system 20 also comprises an anatomical pose resolver 25 for determining a complete target pose CTP of an avatar based on the determined machine-interpretable pose MIP and additionally based on an avatar model PAM from an avatar database 26 including anatomical constraints, wherein physically impossible intersections and collisions of body parts are resolved.

Part of the patient positioning system 20 is further a rendering unit 30a for rendering a fully posed avatar target PAT based on the complete target pose of an avatar CTP.

The patient positioning system 20 includes a human-machine interface 31 for presenting the fully posed avatar target PAT.

The patient positioning system 20 further comprises a non-radiation image capture unit 28 for recording images IM from the patient 33 and a pose detection unit 29 for detecting the current patient pose CPP of the patient 33 using the images IM of the non-radiation image capture unit 28.

Part of the patient positioning system 20 is additionally a comparison unit 30b for comparing the fully posed avatar target PAT with the detected current patient pose CPP of the patient 33.

The patient positioning system 20 comprises an adjustment unit 30c for determining an adjustment measurement ADJ of the medical imaging system 32 which is used for acquisition of medical image data from the patient 33 based on the result R of the comparison as well.

The above descriptions are merely preferred embodiments of the present disclosure but not intended to limit the present disclosure, and any modifications, equivalent replacements, improvements, etc. made within the spirit and principle of the present disclosure should be included within the scope of protection of the present disclosure.

Further, the use of the undefined article “a” or “one” does not exclude that the referred features can also be present several times. Likewise, the term “unit” or “device” does not exclude that it consists of several components, which may also be spatially distributed.

Independent of the grammatical term usage, individuals with male, female or other gender identities are included within the term.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,“ ”connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuity such as, but not limited to, a processor, Central Processing Unit (CPU), a Graphics Processing Unit (GPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C #, Objective-C, Haskell, Go, SQL, R, Lisp, Java®, Fortran, Perl, Pascal, Curl, 0Caml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

What is claimed is:

1. A method for patient positioning for medical image acquisition, the method comprising:
selecting a type of scan for a patient;
selecting a specific human readable target pose template that defines a patient target pose for the type of scan;
determining a machine-interpretable pose based on the specific human readable target pose template using a pose interpreter;
determining a complete target pose of an avatar based on the machine-interpretable pose and based on an avatar model including anatomical constraints using an anatomical pose resolver, wherein physically impossible intersections and collisions of body parts are resolved;
rendering a fully posed avatar target based on the complete target pose of an avatar;
presenting the fully posed avatar target to a user via a human-machine interface;
detecting a current patient pose of the patient using a non-radiation image capture unit;
comparing the fully posed avatar target with the current patient pose of the patient; and
determining an adjustment measurement related to the current patient pose based on a result of the comparing.

2. The method according to claim 1, wherein
the adjustment measurement includes a proposal for physical adjustment of the current patient pose, and
the method includes displaying the proposal using the human-machine interface.

3. The method according to claim 1, wherein determining of the adjustment measurement includes
determining adjusted scan projection parameters of a medical imaging system used for the medical image acquisition, and
adjusting the medical imaging system based on the adjusted scan projection parameters.

4. The method according to claim 3, wherein determining of the adjusted scan projection parameters includes calculating an adjusted scan projection based on the fully posed avatar target or based on the current patient pose of the patient.

5. The method according to claim 1, further comprising:
receiving, by the human-machine interface, amendment information for amending a pose of the fully posed avatar target to generate a corrected fully posed avatar target,
comparing the corrected fully posed avatar target with the current patient pose of the patient, and
determining another adjustment measurement based on a result of the comparing of the corrected fully posed avatar target and the current patient pose of the patient.

6. The method according to claim 5, further comprising:
analyzing the corrected fully posed avatar target using a target interpreter and based on avatar models, and
generating a corrected specific human readable target pose template.

7. The method according to claim 1, wherein the specific human readable target pose template is automatically generated using an anatomically aware large language model applied to a clinical reference free-text description guideline.

8. The method according to claim 1, wherein the avatar model is specified based on at least one of:
gender,
age,
body size,
shape,
height,
weight,
specific anatomical constraints,
maximum joint rotation angulation, or
reach of extremities.

9. The method according to claim 1, wherein specific constraints for a body part used for generating the complete target pose of an avatar are amended based on knowledge of individual disease anomalies of the patient.

10. The method according to claim 1, wherein the avatar model includes a set of avatar models including base poses, used for medical image acquisitions.

11. The method according to claim 1, wherein to provide machine precise positioning, the determining a machine-interpretable pose based on the specific human readable target pose template comprises:
calculating one of a specific scale, an angulation, or a position of each of a body part and skeleton joints of the avatar.

12. The method according to claim 1, wherein determining of the complete target pose of an avatar is performed using one of:
iterative inverse kinematics and collision detection techniques, or
learned AI models from actual human pose data.

13. A patient positioning system, comprising:
a selection unit configured to select a type of scan for a patient;
an anatomically aware large language model unit configured to generate a specific human readable target pose template that defines a patient target pose for the type of scan;
a pose interpreter configured to determine a machine-interpretable pose based on the specific human readable target pose template;
an anatomical pose resolver configured to determine a complete target pose of an avatar based on the machine-interpretable pose and based on an avatar model including anatomical constraints, wherein physically impossible intersections and collisions of body parts are resolved;
a rendering unit configured to render a fully posed avatar target based on the complete target pose of an avatar;
a human-machine interface configured to present the fully posed avatar target;
a pose detection unit configured to detect a current patient pose of the patient using a non-radiation image capture unit;
a comparison unit configured to compare the fully posed avatar target with the current patient pose of the patient; and
an adjustment unit configured to determine an adjustment measurement related to the current patient pose based on a result of the comparison.

14. A non-transitory computer program product comprising instructions that, when executed by a computer, cause the computer to carry out the method of claim 1.

15. A non-transitory computer-readable storage medium comprising instructions that, when executed by a computer, cause the computer to carry out the method of claim 1.

16. The method according to claim 10, wherein the base poses include at least one of
a supine acquisition,
an oblique laying pose on a scanning table,
a standing pose, or
a sitting pose.

17. The method according to claim 3, further comprising:
receiving, by the human-machine interface, amendment information for amending a pose of the fully posed avatar target to generate a corrected fully posed avatar target,
comparing the corrected fully posed avatar target with the current patient pose of the patient, and
determining another adjustment measurement based on a result of the comparing of the corrected fully posed avatar target and the current patient pose of the patient.

18. The method according to claim 4, further comprising:
receiving, by the human-machine interface, amendment information for amending a pose of the fully posed avatar target to generate a corrected fully posed avatar target,
comparing the corrected fully posed avatar target with the current patient pose of the patient, and
determining another adjustment measurement based on a result of the comparing of the corrected fully posed avatar target and the current patient pose of the patient.

19. The method according to claim 4, wherein the specific human readable target pose template is automatically generated using an anatomically aware large language model applied to a clinical reference free-text description guideline.

20. The method according to claim 4, wherein determining of the complete target pose of an avatar is performed using one of:
iterative inverse kinematics and collision detection techniques, or
learned AI models from actual human pose data.
